(19) United States
(12) Patent Application Publication
(10) Pub. No.: US 2016/0338602 A1
(43) Pub. Date: Nov. 24, 2016

(54) DEVICE AND METHOD FOR MEASURING ARTERIAL SIGNALS

(71) Applicant: NOKIA TECHNOLOGIES OY, Espoo (FI)

(72) Inventor: Niku Oksala, Tampere (FI)

(73) Assignee: NOKIA TECHNOLOGIES OY, Espoo (FI)

(21) Appl. No.: 15/111,678

(22) PCT Filed: Jan. 15, 2015
(86) PCT No.: PCT/FI2015/050024

§ 371 Date: Jul. 14, 2016

(30) Foreign Application Priority Data
Jan. 16, 2014 (FI) 20145038

(51) Int. Cl. A61B 5/021; A61B 5/00

(52) U.S. Cl. CPC A61B 5/02125; A61B 5/6825; A61B 5/0261

(57) ABSTRACT

A device (100) for measuring arterial (107) signals, and especially pulse wave velocity, comprises a sensor array comprising a plurality of sensors (101-04) for detecting arterial signals and providing corresponding measuring data. A signal detecting means (106) is used for detecting signal strength of each of said sensors (101-104) separately based on said measuring data of each sensor. A selection logic (108) is used for selecting the measuring data of the sensors providing signals with highest signal strength as a first measuring data (signals responsible of arterial signals), whereupon the device is configured to use said selected first measuring data for determination of pulse wave velocity and wherein measuring data of at least one another sensor not providing said first measuring data is used as a second measuring data.

### TECHNICAL FIELD OF THE INVENTION

The invention relates to a device and method for measuring arterial signals, and especially pulse wave velocity (PWV) measurement. According to an embodiment the invention relates to continuous non-invasive blood pressure measurement system based on the pulse wave velocity measurements.

### BACKGROUND OF THE INVENTION

Arterial signals, such as blood pressure is conventionally measured by devices relying on a tourniquet technology resulting in intermittent measurement. The intermittent measurement has several disadvantages, namely it is slow and cumbersome and in addition it blocks the blood circulation for the measurement. Also some continuous measurement systems are known based on a determination of Pulse Wave Velocity (PWV) and Pulse Transmit Time (PPT) measurements, where the pulse propagating in the blood vessel is detected and based on the wave velocity the blood pressure can be determined. However, the results of these continuous measurement systems are not typically very reliable for example due to changing environmental factors, such as environmental artefacts, motion of the user and motion or positioning of the measuring device in the best position for ensuring reliable signals. In addition during the use the measuring device may also move to an unfavourable position, whereupon the sensors are not measuring signal properly anymore.

### SUMMARY OF THE INVENTION

An object of the invention is to alleviate and eliminate the problems relating to the known prior art. Especially the object of the invention is to provide a device for measuring arterial signals continuously and non-invasively in a reliable, easy and fast way. In addition the object of the invention is to make possible to gather very reliable signal for every measuring cycle taking any surrounding and environmental effect into account, even if the measuring device would move during the use.

The object of the invention can be achieved by the features of independent claims.

The invention relates to a device for measuring arterial signals, especially pulse wave velocity according to claim 1. In addition the invention relates to a corresponding measuring method according to claim 16, as well as to computer program product related claim 21.

According to an advantageous embodiment a device for measuring arterial signals, and especially pulse wave velocity, comprises a sensor array of a plurality of sensors configured for detecting arterial signals and providing corresponding measuring data. The device also comprises signal detecting means for detecting signal strength of each of said sensors separately based on said measuring data of each sensor. In addition a selection logic is used for selecting the measuring data of the sensors providing signals with highest signal strength, advantageously exceeding a certain threshold. The selection can be performed in each continuous measuring cycle, thereby providing an adaptive measurement device.

The selected signals responsible of arterial signals are construed as a first measuring data, and said selected first measuring data is used for determination of pulse wave velocity. Advantageously at least two signals of different sensors are selected for representing said first measuring data. The measuring data of at least one another sensor not selected as said first measuring data is used as a second measuring data and is advantageously construed as representing noise or other artefact data. The first and second sensors selected for representing said first measuring data are arranged to detect the signals so that the first proximal sensor (closest to the heart of the user) detects the signal before the second distal one. This is used as a first quality control so that the signals from other sensors than said first proximal sensor is determined only during a certain time interval triggered by said first signal of the first proximal sensor.

Because the sensors in the array are very close to each other, all the sensors detect essentially the same background noise or other artefacts from the environment. Thus, according to an embodiment said first measuring data including also essentially the same noise data than said second measuring data may be manipulated by said second measuring data in order to eliminate said noise data from the final results, whereupon the maximum correct or reliable signal is derived of the pulse wave after said manipulation. The manipulation is advantageously a mathematical operation, such as a subtraction in an exemplary case.

In the device the sensors are advantageously arranged in an array or matrix, where at least some of the sensors are in a sequence in the longitudinal direction of the device and some of the sensors are arranged in a sequence in the direction essentially perpendicular to said longitudinal direction. The sensor array is advantageously aligned along the course of the radial artery and positioned so that the middle sensor strip is right above the artery while the lateral strips are off the course of the array. This design allows the true arterial signal+noise (random noise+movement artefact) and noise (random noise+movement artefact) to be recorded simultaneously.

According to an embodiment blood pressure is determined based on the pulse wave velocity measurement. The pulse is determined based on the time difference between the first and second detectors of the array detect the same pulse and the distance of said first and second sensors.

In addition according to an embodiment the device comprises also at least one accelerometer, preferably 3D MEMS accelerometer, for measuring movements of the device and thereby the movements of the user. The acceleration data may be used for filtering measuring artefacts due to movements of the device or user so that if the measured data deviates from a predetermined range for a normal state, acceleration data is determined. If the acceleration data is normal in the case the measured data deviating from a predetermined range, there might be a problem relating to the user's health. Instead if the acceleration data implies that the user for example runs or jumps, the measurement data is compared to a predetermined range for an active state. In addition, if the measured data is out of normal range and the acceleration data reveals abnormal accelerations due to environmental factors, such as traffic vibration or the like, the deviated measured data may be ignored, for example.

Furthermore the acceleration data may also be used for calibration of the device by measuring different position of the device or actually different positions of the arm (upper extremity) of the user, namely in different positions different measurement results are achieved due to e.g. changing hydrostatic pressures in the blood vessels. An example of the calibration procedure is described elsewhere in this document. The calibration may be performed as a continuous routine.

The sensors used may be capacitive sensors, passive IR sensors, photo-plethysmography sensors (PPG), CCD sensor or EMFI (electromechanical film) sensors. Most advantageously optical sensors are used, since they best allow movements of the sensor device and they are not very sensitive for example for environmental artefacts. The device advantageously comprises 3-16 sensors, but it is clear that also more sensors may also be used.

The present invention offers advantages over the known prior art, such as continuous measurements of the arterial signals, such as pulse wave velocity and thereby blood pressure. In addition the signals may still be measured even if the user is moving or even if the device is moved over the artery. Moreover also environmental factors may be taken into account and thereby ensuring reliable signals. Furthermore the invention offers also the possibility to perform continuous and non-invasive blood pressure measurements. This is based on pulse wave velocity (PWV) measurement with continuous automatic calibration. Especially it is to be noted that measurements can be done without any direct blood pressure measurements, such as tourniquet techniques or sensors which should be pressed tightly against the body, which offers clear advantages.

### BRIEF DESCRIPTION OF THE DRAWINGS

Next the invention will be described in greater detail with reference to exemplary embodiments in accordance with the accompanying drawings, in which:

FIGS. 1A-1E illustrate a principle of an exemplary device for measuring arterial signals continuously and non-invasively according to an advantageous embodiment of the invention,

FIGS. 2A-2B illustrate another exemplary layout of sensors of the device for measuring arterial signals continuously and non-invasively according to an advantageous embodiment of the invention, and

FIG. 3 illustrates exemplary usage of the device according to an advantageous embodiment of the invention.

### DETAILED DESCRIPTION

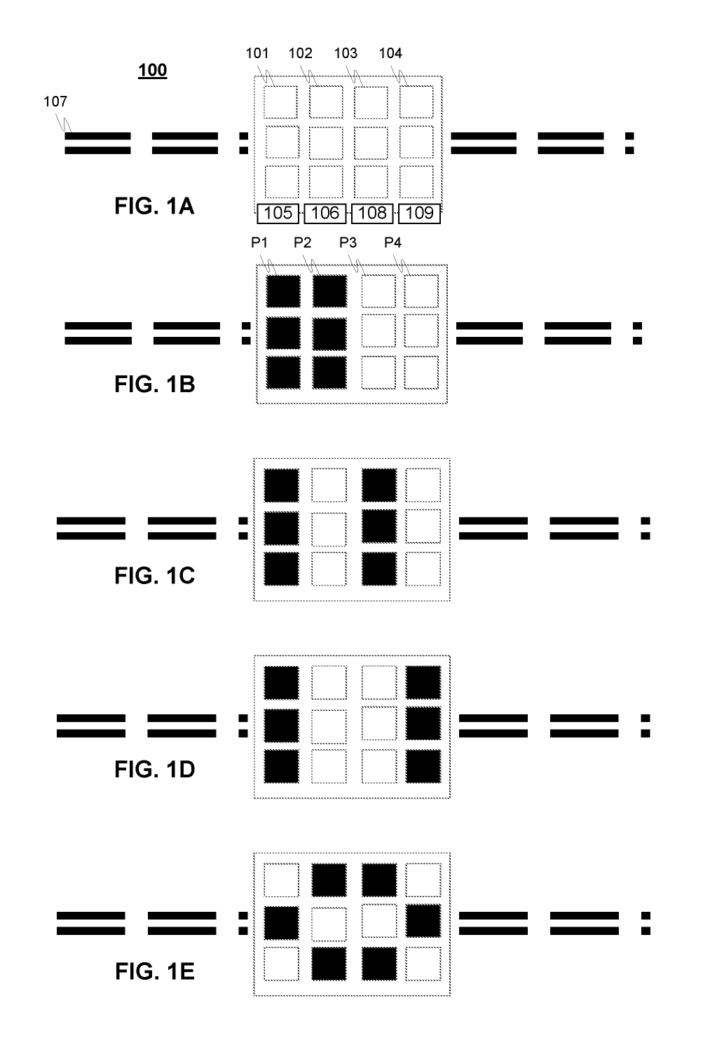

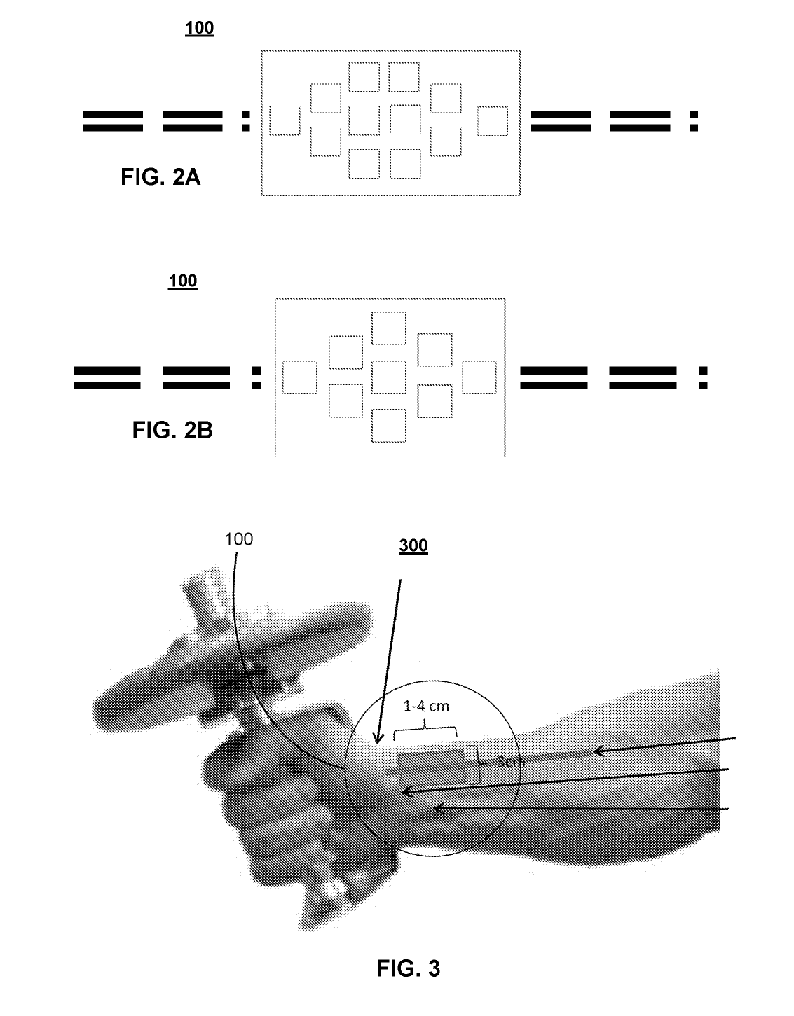

FIGS. 1A-1E illustrate a principle of an exemplary device 100 for measuring arterial signals continuously and non-invasively according to an advantageous embodiment of the invention, where the device comprises a sensor array (matrix) comprising a plurality of sensors 101, 102, 103, 104 for detecting arterial signals and providing corresponding measuring data.

In the device at least some of the sensors are arranged in sequence in the longitudinal direction of the device and some of the sensors are arranged in sequence in the direction essentially perpendicular to said longitudinal direction so that advantageously at least two of said sensors are always located on the artery 107. Advantageously the sensor array is configured to be aligned along the course of distal radial artery 107.

The device also comprises signal detecting means 105 for detecting signal strength of each of said sensors separately based on said measuring data of each sensor, as well as a selection logic 106 for selecting the measuring data of the sensors providing signals with highest signal strength as a first measuring data (signals responsible of arterial signals measured from the artery 107). The device is configured to use the selected first measuring data for determination of pulse wave velocity. The measuring data of at least one another sensor not providing said first measuring data is used as a second measuring data.

Due to the array or matrix form of the sensors the first sensor 101, P1 producing first a signal with strength exceeding a threshold is determined the sensor as closest to the heart of the user. This signal can be used as a trigger for triggering a time interval during which any measuring signals from other sensors 102-104 are determined. The signal from at least one other sensor 102, P2 is used as said first measuring data (together with the signal from the first sensor 101, P1), if the second signal strength also exceeds a threshold. It is to be noted that also other requirements may be required, such as signal form must be matched to a predetermined form or shape or also the amplitude of the second signal should be smaller than the amplitude of the signal produced by said first sensor so that said second signal 102, P2 is qualified as said first measuring data.

In addition signals from at least one other sensor 103, P3, 104, P4 is used as said second measuring data and construed as representing noise (or other artefact) data. It is to be noted that because the sensors are very close to each other also said first measuring data from sensors 101, P1, 102, P2 includes also essentially the same noise data than said second measuring data from sensor 103, P3, 104, P4. In order to achieve reliable measuring data said first measuring data is advantageously manipulated by said second measuring data in order to eliminate said noise data.

It is to be noted that advantageously signals from all sensors 101-104 are determined and only the signals exceeding the threshold (strongest signals from the sensors locating above the artery 107 or at least next to the artery 107) is selected for said first measuring data.

As can be seen in FIG. 1E the sensors painted black are providing the best signal strength and thus they are selected as representing the first measuring data, whereas signal from at least one other sensor (painted white) essentially not producing any arterial based signal is used for said second measuring data representing essentially only the background noise or other artefact signal.

According to an embodiment the sensors are configured to measure the arterial based signals, such as optically measurable signals due to arterial blood pressure changes of a user, at certain locations. For deriving blood pressure the device 100 or any other backend system advantageously comprises data processing means 108 for determining blood pressure from the measured signals. For this the selection logic selects measurement data of at least one first and one second sensor as representing said first measurement data so that said first sensor (P1) is configured to measure said signal at a first location and said second sensor (P2) is configured to measure said signal at a second location in order to derive pulse wave velocity. The blood pressure is determined based on the pulse wave velocity measurement, wherein the velocity of the pulse is determined based on the time difference between the first and second sensors of the array detect the same pulse and the distance of said first and second sensors.

According to an embodiment the first and second sensors (as well as also other sensors) are arranged in the device so that in use they are configured to be positioned against measurement location of a user at a known fixed distance from each other, wherein the distance is between 0.5-5 cm, more advantageously between 1-4 cm, for example. Still according to an example sampling resolution of the sensors may be a magnitude of at 100 Hz, more advantageously at least 1 kHz.

It is to be understood that the data processing, such as manipulation of the first measurement data with said second measurement data as well as also other signal or data processing (108) may be performed in backend system (not shown), whereupon the device comprises advantageously wireless data communication means for communicating measurement signal to the backend. Therefore also signal detecting means 105 and/or the selection logic 106 may also be implemented by the backend system. In addition it is to be noted that the device may also comprise at least one accelerometer 109.

FIG. 3 illustrates exemplary usage 300 of the device according to an advantageous embodiment of the invention.

The sensor array is advantageously aligned along the course of the radial artery (107) and positioned so that the middle sensor strip is right above the artery while the lateral strips are off the course of the array. This design allows the true arterial signal+noise (random noise+movement artefact) and noise (random noise+movement artefact) to be recorded simultaneously. According to an example the sensor array may comprise preferably 3 pieces of 1×4 EMFI-sensor strips, in which all the individual sensors are separately wired. Also other types of sensors can be utilized. This design offers more reference sensor resolution in lateral dimension and allows easier manipulation of proximal-distal distance

According to an example the device 100 may comprise at least one, preferably two accelerometers 109 for detecting movements of the user, such as movements of the hand or other changes in altitude, i.e. falls and collapses. The device may be configured to detect these movements based on the changes in detected pressure signals possibly supplemented by the measurements of said accelerometers, or alternatively based signals purely detected by said accelerometers. The accelerometers are advantageously 3D MEMS accelerometers. It is to be noted that the device additionally comprises also other components allowing the measurements, such as an MCU or ASIC logic circuit (logic, 108), power source, like a battery, or the like.

For measuring blood pressure of a patient continuously and non-invasively according to advantageous embodiments of the invention, the next method steps may be performed by the device.

Utilizing signal processing system, the sensors P1, P2 are selected so, that maximum signal strength is derived and that both arterial pressure sensors P1, P2 detect the signals so that the proximal sensor fires before the distal one. This procedure provides the first quality control.

According to an exemplary embodiment also a third capacitive pressure sensor may be utilized to measure the ambient pressure signal. The signal derived from this ambient pressure sensor may be subtracted from signals derived from the arterial sensors P1, P2 to compensate for alterations induced by alterations in measurement point altitude (i.e. postural changes, alterations in measurement point position relative to heart) and atmospheric pressure changes. This signal can yield changes in altitude with a resolution of centimetres and therefore measure the changes in the vertical position of the arterial pressure sensors. For example, if the ambient pressure suddenly rises or decreases (i.e. during movement of arm, climbing of stairs or opening or closing of doors), this is immediately reflected also in the arterial sensor readings and amplitude of the pulse wave.

Utilizing the embodiments of the invention the signal to noise ratio can be maximized continuously. For example, raising the hand above the head results in greatly lowered amplitude of the pulse wave in addition to obvious slowing down of the PWV. This makes it hard to reliably detect the critical phases of the wave (i.e. the foot-phase of the pulse wave) needed for accurate PWV calculation. One of the primary interests of the invention is to derive the systemic arterial pressure of which the pressure reading at the wrist is an approximation. The movement of the hand can be detected by the accelerometer. The accelerometer reading can also be used to extrapolate the systemic pressure since in addition to the initial calibration procedure (see below, yielding the distance from heart level to wrist area) it makes it possible to continuously detect the changes in measurement point height during patient movement and compensate the readings accordingly. It can also be utilized to model rapid changes in altitude, i.e. falls and collapses.

In addition, according to an embodiment movements of the hand or other changes in altitude, i.e. falls and collapses, can be additionally or independently detected by accelerometers (such as 3D MEMS accelerometers), which can be configured to be capable of detecting upper arm movements and providing signals indicating walking, standing, sitting and laying supine, as an example.

### Baseline Calibration Procedure

The accelerometer or additional ambient pressure sensor can be used for baseline calibration. Blood pressure measurement should be performed so that the measurement point stays at a constant distance from heart. The accelerometer or ambient pressure sensor can yield the change in vertical displacement or altitude relative to sea level at a resolution of few centimeters as atmospheric pressure is a function of altitude. Therefore, the system automatically calibrates to different measurement conditions, regardless of altitude. This provides a second quality control (C2). To convert relative measures to absolute ones, a patient specific calibration procedure is performed so that when lying supine, the upper limb is raised or flexed straight at an angle of 90° relative to the horizontal plane. This procedure can be monitored, according to an exemplary embodiment, by the accelerometers (e.g. 3D MEMS accelerometers) and the PWV calculation algorithm is executed when the 90° angle is achieved. Using the equation (1), where Δh is the altitude change, ρ is the density of blood which is considered constant and g is the gravitational constant the absolute change in hydrostatic pressure (ΔP_{hydrostatic}) calculated:

ΔP_{hydrostatic}=Δhρg (1)

Using this equation, the pressure values from arterial sensors can be calibrated to absolute values. This provides a third quality control (C3). This procedure also yields the approximate distance Δh from body to wrist to be utilized in continuous auto calibration sequences. The changes in ambient temperature in this context are considered not significant. To yield another, potentially more reliable measure of arterial pressure, two other parameters are derived. The time needed (i.e. pulse transit time PTT) for the pulse wave to propagate from proximal sensor to distal sensor (P1, P2) is calculated by a mathematical algorithm tracking a specific point at the foot of the pulse wave known to be insensitive to reflections of the pulse wave. The result is the pulse wave velocity (PWV) and PTT. Alterations in PWV and PTT have been shown to correlate well with alterations in systemic arterial pressure. However, interpersonal correlation is weaker. The signal processing algorithm may be integrated in the signal processing unit of the component itself or located in a remote backend system.

The absolute pressure values are derived by first utilizing the Moens-Korteweg equation (2), where t is the thickness of the artery wall, d is the diameter of the artery, ρ is the density of blood which is considered constant, and E is the Young's modulus reflecting the elasticity of the arterial wall. This equation can also be used to derive E, a parameter which associates with probability of future cardiovascular events when PWV is known:

$$P W V = \sqrt{\frac{tE}{ρ d}} \qquad ( 2 )$$

The Young's modulus E is not constant but varies with pressure. The dependence of E on pressure is shown by equation (3), where E₀ is the zero pressure modulus, α is a vessel constant (experimentally validated α=0.017 mmHg⁻¹), P is pressure and e is the Euler number (2.71828 . . . ):

E=E₀e^{αP} (3)

When equation (2) is substituted to (3) it yields equation (4) which describes the association of PWV with P and zero pressure elasticity E₀.

$$P W V = \sqrt{\frac{{tE}_{0} {e}^{α P}}{ρ d}} \qquad ( 4 )$$

From this equation, P can be solved:

$$P W {V}^{2} = \frac{{tE}_{0} {e}^{α P}}{ρ d} \qquad ( 5 )$$

Of specific importance is that from this equation E₀ or subsequently E can also be solved then describing the association of zero pressure elasticity or Young's modulus E and PWV when pressure P is known, derived either by external measurement device or previously described method (A) which can be utilized with adequate accuracy at least when the measurement is performed under constant mounting pressure conditions (E₀=PWV²ρd/[te^{αP}] or E=PWV²ρd/t). These parameters can be utilized in the prediction of future cardiovascular events or in the monitoring of treatment response.

$$\begin{matrix}\frac{ρ d P W {V}^{2}}{{tE}_{0}} = {e}^{α P} & ( 6 ) \\ ln ( \frac{ρ d}{{tE}_{0}} P W {V}^{2} ) = ln {e}^{α P} & ( 7 ) \\ ln ( \frac{ρ d}{{tE}_{0}} P W {V}^{2} ) = α P & ( 8 ) \\ ln ( \frac{ρ d}{{tE}_{0}} ) + ln ( P W {V}^{2} ) = α P & ( 9 ) \\ ln ( \frac{ρ d}{{tE}_{0}} ) + 2 ln ( P W V ) = α P & ( 10 )\end{matrix}$$

Of specific importance is that from equation (10) α can be easily solved when P and PWV are known.

$$\begin{matrix}P = \frac{1}{α} ln ( \frac{ρ d}{{tE}_{0}} ) + \frac{2}{α} ln ( P W V ) & ( 11 ) \\ P = K + \frac{2}{α} ln ( P W V ) & ( 12 ) \\ with & \\ K = \frac{1}{α} ln ( \frac{ρ d}{{tE}_{0}} ) & ( 12 )\end{matrix}$$

From the equation (12) one can see that pressure is easily derived taken that the constant K is obtained. During the calibration procedure, equation (1) holds and the absolute value of ΔP_{hydrostatic} is known since Δh is directly obtained from the ambient pressure sensor (or from the accelerometer data, as is disclosed elsewhere in this document):

ΔP_{hydrostatic}=Δhρg (1)

During calibration procedure, the hydrostatic pressure changes when the upper limb is raised. Substituting equation (1) into equation (12) yields:

$$\begin{matrix}Δ {P}_{hydrostatic\_calibration} = K + \frac{2}{α} ln ( Δ P W {V}_{calibration} ) & ( 13 ) \\ K = Δ {P}_{hydrostatic\_calibration} - \frac{2}{α} ln ( Δ P W {V}_{calibration} ) & ( 14 )\end{matrix}$$

Therefore, the patient-specific and measurement-specific constant K can be obtained during the calibration procedure. The optimal procedure is to first determine K during calibration procedure using equation (14), then substituting K into equation (12) giving the pressure P as a function of PWV.

$$P = Δ {P}_{hydrostatic\_calibration} - \frac{2}{α} ln ( Δ P W {V}_{calibration} ) + \frac{2}{α} ln ( P W V ) \qquad ( 15 )$$

Changes in the position of the upper limb relative to body cause alterations in hydrostatic pressure. These changes can be compensated easily since the accelerator or ambient pressure sensor continuously reports the changes in height. These considerations apply only when the system is used at constant altitude since there is no body reference altitude sensor. Therefore, the system may be built so that the equation (15) is substituted with a hydrostatic pressure term (ΔP_{hydrostatic}_{_}_{calibration}) correcting for upper limbposition alterations relative to heart. This term is either positive or negative depending on the altitude change relative to default set point determined during baseline calibration:

$$P = Δ {P}_{hydrostatic\_calibration} - \frac{2}{α } ln ( Δ P W {V}_{calibration} ) + \frac{2}{α} ln ( P W V ) + Δ {P}_{hydrostatic\_position} \qquad ( 16 )$$

It is to be noted that the baseline calibration procedure yielding Δh and ΔP_{hydrostatic}_{_}_{calibration} and subsequently ΔPWV_{calibration} can be done utilizing the two accelerometers. According to an embodiment this can be implemented even without the ambient pressure sensor. For example, as one of the three 3D accelerometer axes in both accelerometers is positioned perpendicular to the wristband or device and parallel to axis of the upper limb, it is therefore capable of measuring the centrifugal or radial accelerations a₁ and a₂ at distances r₁ (the proximal accelerometer) and r₂ (the distal) along the axis of the upper limb.

In the following equation, the radial accelerations at the specified two measurement locations where ω is the angular velocity are:

a₁=ω²r₁ and a₂=ω²r₂ (17)

The difference in acceleration between the two accelerometers is:

a₂−a₁=ω²r₂−ω²r₁ (18)

Subsequently, let D be the fixed distance between the two accelerometers

(D=r₂−r₁):

a₂−a₁=ω²(r₂−r₁) (19)

which yields the angular velocity of the upper limb:

ω=[(|a_{g}−a₁|)/D]^{1/2} (20)

The radius r=(r₂+r₁)/2 at the center of the wristband which equals Δh when the upper limb is flexed or raised at 90° angle relative to the vertical axis of the patient when standing erect or sitting, i.e. strictly horizontally, can then be calculated. The centrifugal force at the center of the wristband during rigorous horizontal swing of the upper limb can be calculated:

F=(mω²)/r (21)

r=(mw²)/F, (22), where F=ma, and m is the mass of the accelerometer sensor element which is the same in both accelerometers and therefore their average is simply m, where a is the acceleration (a₂+a₁)/2 at the center of the wristband

r=ω²/a (23)

Substituting equation (20) into (23) yields:

r=[(|a₂−a₁|)/D]/a, (24)

r=[(|a₂−aᵢ|)/D]*2/(a₂+a₁) (25), and r=Δh

r=2(|a₂−aᵢ|)/[D(a₂+a₁)] (26)

Subsequently, when the upper limb is flexed at 90° position relative to the plane when the patient is lying supine, the ΔPWV_{calibration} is recorded simultaneously with ΔP_{hydrostatic}_{_}_{calibration} and the values processed as described before.

Utilizing the pulse wave curve, an algorithm can be utilized to derive heart rate as number of pulse waves per time unit, respiratory rate from baseline, amplitude and heart rate variability using wavelet transform function.

### Continuous Auto Calibration Procedure

The subtraction of ambient pressure reading from pressure dericed from P1 and P2 results in stable amplitude and maximal signal-to-noise ratio. The readings from ambient pressure can be used to detect changes measurement point altitude and therefore movement of wrist relative to heart level during movement or postural changes. This data can also be used to extrapolate systemic pressure levels as described earlier since the Δh is obtained during baseline calibration sequence.

The readings from ambient pressure can be used to extrapolate systemic pressure levels or compensate for movement or postural changes. It is to be noted that the changes in the ambient pressure due to height variations can be extrapolated by using accelerometer data as described above.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims. For example it is to be noted that, analogously as in the baseline calibration procedure, the accelerometer sensor output yielding the angular velocity w and tilt of the upper limb can be used for continuous autocalibration. In addition it is to be noted that the accelerometers described above may be e.g. 3D MEMS accelerometer or similar known from the prior art.

In addition it is to be noted that the device for measuring arterial signals, and especially pulse wave velocity, can be advantageously implemented by a wristband device, where the wristband device comprises advantageously all sensors. The data processing can be implemented by the wristband device, or alternatively the wristband device may send (e.g. wireless way) the measuring signals to the external data processing backend for data calculation. The data processing backend may comprise e.g. could server, any computer or mobile phone application and according to an example it can send the calculated results or otherwise processed data e.g. for displaying back to the wristband device or other data displaying device, such as a computer or the like in data communication network or to a smartphone of the user.

1. A device,
comprising:
a sensor array comprising a plurality of sensors configured to detect arterial signals and provide corresponding measuring data,
a signal detector configured to detect a signal strength of each of said plurality of sensors separately based on said measuring data of each sensor, and
selection logic configured to select measuring data of sensors of the plurality of sensors providing signals with a highest signal strength as a first measuring data, wherein the device is configured to use said selected first measuring data for determination of pulse wave velocity and wherein measuring data of at least one other sensor not providing said first measuring data is used as a second measuring data,
wherein
the device is configured to determine a first sensor producing first a first signal with a strength exceeding a threshold as a sensor closest to a heart of a user, and wherein the device is configured to determine a second signal with a strength exceeding a threshold from at least a second sensor during a certain time interval triggered by said first signal.

2. A device of claim 1, wherein said second measuring data is construed as representing noise data, and wherein said first measuring data including essentially the same noise data as said second measuring data is manipulated by said second measuring data in order to eliminate said noise data.

3. A device of claim 1, wherein the device comprises at least one accelerometer for measuring movements of the device and thereby the movements of the user, and wherein at least one of:
acceleration data is used for filtering measuring artefacts due to movements of the device so that if the measuring data deviates from a predetermined range, acceleration data is determined and if there is an abnormal acceleration, the deviated measuring data is filtered; or
acceleration data is used for calibration of the device via measuring positions of the device.

4. A device of claim 1, wherein at least some of the plurality of sensors are arranged in sequence in a longitudinal direction of the device and some of the plurality of sensors are arranged in sequence in a direction essentially perpendicular to said longitudinal direction.

5. A device of claim 1, wherein a signal corresponding to said measuring data of said at least one other sensor is used as said first measuring data if at least one of a signal form of said signal corresponding to said measuring data of said at least one other sensor matches a predetermined form shape or an amplitude of said signal corresponding to said measuring data of said at least one other sensor is smaller than an amplitude of the first signal produced by said first sensor.

6. A device of claim 1, wherein the plurality of sensors are at least one of optical sensors, capacitive sensors, passive IR sensors, photo-plethysmography sensors, CCD sensors or EMFI sensors, and wherein the device comprises 3 to 16 sensors.

7. A device of claim 1, wherein said plurality of sensors are configured to measure said arterial signals, at a certain location, and wherein the device is configured to select measuring data of at least one said first sensor or said second sensor as representing said first measurement data so that said first sensor is configured to measure said first signal with a strength exceeding a threshold at a first location and said second sensor is configured to measure said second signal with a strength exceeding a threshold at a second location.

8. A device of claim 1, wherein the first and second sensors are arranged in the device so that in use they are configured to be pressed against a measurement location of said user at a known fixed distance from each other, wherein the distance is one of between 0.5-5 cm or between 1-4 cm.

9. A device of claim 1, wherein the sensor array is configured to be aligned along a course of a distal radial artery.

10. A device of claim 1, wherein the selection logic is configured to select the measuring data of sensors of the plurality of sensors providing signals with a highest signal strength separately for each continuous measuring cycle and thereby provide an adaptive measuring device.

11. A device of claim 1, wherein a sampling resolution of the plurality of sensors is a magnitude of one of 100 Hz or at least 1 kHz.

12. A device of claim 2, wherein a maximum signal is derived of a pulse wave after said manipulation and wherein the first and second sensors are arranged to detect the signals exceeding a threshold so that a first proximal sensor of said first and second sensors detects a signal exceeding a threshold before a second distal sensor of said first and second sensors detects a signal exceeding a threshold.

13. A device of claim 1, wherein blood pressure is determined based on pulse wave velocity measurement, and wherein a velocity of a pulse is determined based on a time difference between the first and second sensors of the array detecting a same pulse and a distance of between said first and second sensors.

14. The device of claim 1, wherein the device further comprises a third ambient pressure sensor a signal of which is used for calibration of measurements of at least one of the first or second sensors so that signals representing an absolute systemic arterial blood pressure of the user are provided.

15. The device of claim 1, wherein the device is implemented by a wristband device, wherein the wristband device comprises said sensor array comprising a plurality of sensors configured to detect arterial signals and provide corresponding measuring data,
wherein the wristband device is configured to one of:
detect signal strength of each of said sensors separately based on said measuring data of each sensor, and
select the measuring data of the sensors of the plurality of sensors providing signals with a highest signal strength as a first measuring data, wherein the device is configured to use said selected first measuring data for determination of pulse wave velocity and wherein measuring data of at least one other sensor not providing said first measuring data is used as a second measuring data,
or
send said measuring data to a backend data processing unit for determination of one of arterial signals or pulse wave velocity.

16. A method, comprising:
providing a sensor array comprising a plurality of sensors configured to detect arterial signals and provide corresponding measuring data,
detecting a signal strength of each of said plurality of sensors separately based on said measuring data of each sensor, and
selecting measuring data of sensors of the plurality of sensors providing signals with a highest signal strength as a first measuring data, wherein the device is configured to use said selected first measuring data for determination of pulse wave velocity and wherein measuring data of at least one other sensor not providing said first measuring data is used as a second measuring data₌
wherein
a first sensor producing first a first signal with strength exceeding a threshold is determined as a sensor closest to a heart of a user, and wherein a second signal with strength exceeding a threshold is determined from at least a second sensor during a certain time interval triggered by said first signal.

17. A method of claim 16, wherein said second measuring data is construed as representing noise data, and wherein said first measuring data including also essentially the same noise data as said second measuring data is manipulated by said second measuring data in order to eliminate said noise data.

18. A method of claim 16, wherein at least one accelerometer is used for measuring movements of the device and thereby the movements of the user, and wherein at least one of:
acceleration data is used for filtering measuring artefacts due to movements of the device so that if the measuring data deviates from a predetermined range, acceleration data is determined and if there is an abnormal ccelerations acceleration, the deviated measuring data is ignored; or
acceleration data is used for calibration of the device via measuring position of the device.

19. A method of claim 17, wherein a maximum signal is derived of a pulse wave after said manipulation and wherein the first and second sensors are arranged to detect the signals exceeding a threshold so that a first proximal sensor of said first and second sensors detects a signal exceeding a threshold before a second distal sensor of said first and second sensors detects a signal exceeding a threshold.

20. A method of claim 16, wherein blood pressure is determined based on pulse wave velocity measurement, and wherein a velocity of a pulse is determined based on a time difference between the first and detesters sensors of the array detecting a same pulse and a distance of between said first and second sensors.

21. (canceled)
